(19) United States
(12) Patent Application Publication
(10) Pub. No.: US 2022/0367047 A1
(43) Pub. Date: Nov. 17, 2022

(54) METHOD FOR SIMULATING THE DEFORMATION, AFTER IMPLANTATION, OF AN IMPLANTABLE MEDICAL DEVICE

(71) Applicant: SIM&CURE, MONTPELLIER (FR)

(72) Inventors: Julien SIGÜENZA, MONTPELLIER (FR); Dominique AMBARD, Aniane (FR); Christophe CHNAFA, MONTPELLIER (FR)

(21) Appl. No.: 17/765,134

(22) PCT Filed: Oct. 16, 2020
(86) PCT No.: PCT/FR2020/051864

§ 371 Date: Mar. 30, 2022

(30) Foreign Application Priority Data
Oct. 18, 2019 (FR) FR1911706

(51) Int. Cl. G16H 50/00; B29C 45/76

(52) U.S. Cl. CPC G16H 50/00; B29C 45/76; B29C 45/7693

(57) ABSTRACT

The invention relates to a method for simulating the deformation of an IMD after implantation in a natural cavity, from a three-dimensional model of a wall of the cavity, comprising the steps of:
determination of an intermediate deformation state of a numerical IMD, deformed as a function of a shape of the wall model while remaining included in said shape,
calculation of a mechanical equilibrium state of the numerical IMD from the intermediate deformation state, comprising the calculation of mechanical stresses undergone by the numerical IMD in the intermediate deformation state which are a function of the mechanical behaviours of the numerical IMD and the wall model, and relaxation of said stresses,
the behaviour of the wall model being taken as non-deformable rigid during the calculation of the mechanical equilibrium state,
the mechanical behaviour of the numerical IMD, and/or the rest state of the numerical IMD, being different between the determination of the intermediate deformation state and the calculation of the mechanical equilibrium.

### TECHNICAL FIELD OF THE INVENTION

The present invention pertains to the field of numerical simulation of implants in a natural cavity, before or during the implantation operation.

### PRIOR ART

It is common to use an implant of expandable implantable medical device (IMD) type, such as a “stent”, an “intrasaccular cage”, or a “flow diverter”, to treat for example an artery affected by an aneurysm. It is sought to avoid the expansion and the rupture of the aneurysm. It is furthermore sought to avoid blood clots formed in the aneurysmal sac migrating and locally blocking an artery.

It is advantageous for a health practitioner who has at his disposal a three-dimensional mapping of an artery undergoing a local pathology such as an aneurysm to predict the final shape and position that will be taken by an implant after deployment of said implant in the artery. The prediction of the deformation of the implant is preferably carried out before the insertion of said implant by endovascular route. Thus, the practitioner can select a suitable implantable medical device (hereafter IMD) reference, with an optimal size and an optimal initial positioning of the device.

The planning of the placement of an implant by endovascular route is generally done by means of two-dimensional or three-dimensional images of the artery. Until now, the choice of the IMD reference to implant is based a minima on planar local measurements (two-dimensional) carried out by the physician on images taken of the patient. These measurements do not make it possible to predict an apposition of the IMD in a reliable manner, nor its shape, nor its final position after deployment.

In addition, planar local measurements are based on the experience of the physician who does not benefit from computer assistance for these measurements, which induces great variability in the choice of the IMD to implant.

On the basis of planar local measurements, several manufacturers of implantable medical devices have proposed the use of nomograms or predetermined mathematical relationships to guide practitioners in the selection of the IMD to use for a given patient. It is possible to simulate the final shape of the IMD and to characterise an IMD reference that is suited to the morphology of the natural cavity.

The use of nomograms has however limits. The design of nomograms (for example, in the form of a relationship between the diameter of the natural cavity of the patient and the predictive length of the IMD after deployment in the cavity) is mainly based on empirical observations.

The international patent application WO 2019/122665 A1, in the name of the Applicant, describes a method for simulating the final length of an IMD, notably of a “flow diverter” type implant, in a natural cavity after its deployment. The IMD is discretised into a set of longitudinal three-dimensional segments, for which a cylindrical shape is simulated, then the diameter of each of the segments is modified in an iterative manner while taking into account the geometric stresses exerted by the walls of the cavity on the IMD.

Even though this document indicates that a mechanical modelling of the segments may be adopted before predicting the final length, this method of the prior art remains limited to the prediction of the longitudinal deformation and the apposition of an IMD. It is based on the calculation of the geometric stresses exerted by the walls of the cavity.

This method of the prior art does not make it possible to calculate a field of deformations on the mechanical elements of the IMD, after implantation of the latter in the cavity. A mechanical equilibrium for the IMD after deployment is not calculated.

In addition, this simulation method has good precision for implants of “flow diverter” type but it is not very suited for implants of intrasaccular cage or “laser-cut stent” type of general non-cylindrical shape.

Furthermore, the methodology described in this document works in an optimal manner if the vascular cavity is considered as a succession of straight cylindrical portions with variable radii. As a first approximation, it is considered that the changes in configurations of successive portions of the IMD model preserve the cylindrical shape of these portions, even if an alternative of the method described in this document proposes taking into account a parameter of longitudinal compression exerted on the IMD during implantation in the vascular cavity.

The publication Patient-specific numerical simulation of stent-graft deployment: Validation on three clinical cases, Perrin et al., HAL Id: inserm-01201545, 17 Sep. 2015, describes a simulation method making it possible to predict the final deployed shape of an arterial endoprosthesis of general cylindrical shape. A three-dimensional geometry of the artery surface is obtained by means of a shell element model by interpolation, the artery wall being modelled with an orthotropic linear elastic behaviour. A behaviour of the implant is also modelled as orthotropic elastic. This document describes a radial compression of the implant model, then an insertion of the implant model in the artery wall model with boundary conditions which avoid simulating the complete deployment of the implant model.

However, the calculation of the static mechanical equilibrium between the implant model and the artery wall model remains laborious, and is liable to generate instability modes as a function of the shape of the IMD and the artery. In particular, the methodology described in this document does not seem to be well suited for other types of IMD, such as an intrasaccular cage.

The precision of the prediction of the final shape of the IMD after its deployment may thus be further improved and no satisfactory prediction method exists for IMDs which are not of the “flow diverter” type. In the case of an intrasaccular or intra-aneurysmal cage with variable geometry, the implant can expand longitudinally and radially around its axis of revolution in the aneurysmal sac after implantation. The implant hugs the shape of the aneurysmal sac after expansion, up to mechanical equilibrium with the wall of the aneurysmal sac. The simulation methods of the prior art do not take account of these deployment particularities of the implant.

### DESCRIPTION OF THE INVENTION

With regard to the preceding, there exists a need for a method for simulating the final position and deformations of an implantable medical device (hereafter IMD) in a natural cavity, which can be implemented before any intervention on a patient to implant the IMD. The sought after method must provide results of deformation of the IMD after implantation which are close to the final position actually observed, in order to reach a sufficient degree of clinical precision while being adaptable to a large variety of patients and interventions.

The sought after method must further be rapid and not demanding in calculation time, so as to converge rapidly towards a simulation result (mechanical equilibrium state of the IMD) which is useful for the choice of the IMD and the implantation conditions by the practitioner, notably in an emergency situation. An order of magnitude of the desired calculation time, once the input data selected and for a given IMD reference, is for example from 5 to 60 seconds.

In addition, the sought after method must be robust for the widest possible range of IMDs, for example for IMDs of non-tubular shape such as braided cages, while providing stable numerical solutions. None of the methods of the prior art gives satisfaction for this type of IMD shape.

There exists an additional need for a method for simulating the final position and shape of the IMD which makes it possible to predict the local apposition of the implant on the wall of the natural cavity.

In this respect, the invention relates, according to a first aspect, to a method for simulating a deformation after implantation of an implantable medical device, called IMD, in a natural cavity, from a three-dimensional numerical model of a wall of the cavity, the method comprising the following steps implemented by a processing unit:

i. determination of an intermediate deformation state of a numerical IMD representing the IMD, the numerical IMD at the intermediate deformation state being deformed as a function of a shape of the wall model while remaining included in said shape,

ii. calculation of a mechanical equilibrium state of the numerical IMD from the intermediate deformation state, comprising the calculation of mechanical stresses undergone by the numerical IMD in the intermediate deformation state which are a function of a mechanical behaviour of the numerical IMD and a mechanical behaviour of the wall model, and comprising the relaxation of said stresses, the calculated mechanical equilibrium state corresponding to the simulated deformation of the IMD after implantation.

The simulation method of the invention enables the health practitioner to obtain a prediction of the final state of an IMD implanted in a natural cavity of a patient. This prediction takes account of the predictive mechanical behaviour of the IMD and the behaviour of the wall of the natural cavity, enabling a precise simulation.

The determination of the intermediate deformation state, before calculating a mechanical equilibrium state, makes it possible to simplify the calculation of the mechanical equilibrium because it provides an efficient initialisation of said calculation. A large number of methods for simulating IMD deformation of the existing scientific literature reproduce virtually the whole of the real deployment of the IMD, initially compressed in a micro-catheter. The IMD adopts, during its real deployment, a “history of deformations” which is highly complex to simulate. These methods thus prove costly in calculation times and are sometimes not very stable, making them difficult to be compatible with clinical practice.

The simulation method of the invention does not require reproducing the entire real history of deformations, and proposes defining an intermediate deformation state (theoretical) to accelerate and simplify the simulation of deployment of the IMD.

The simulated numerical IMD adopts, in the course of the method of the invention, a simplified “non-physical” history of deformations, which is easier to simulate (faster and more stable simulation than the simulation of the complete history of deformations). This simplified history of deformations preferably starts with a rest state of the numerical IMD. The IMD then goes through an intermediate deformation state, then the mechanical stresses undergone by the IMD in the intermediate deformation state are taken into account and the relaxation of these stresses is simulated, to reach a final deformed state of the IMD.

The final deformed state of the IMD obtained with this method remains close to reality. The intermediate deformation state is chosen shrewdly so as to simulate efficiently the entire history of deformations. Notably, it is possible to choose a specific mechanical behaviour of the numerical IMD during the determination of the intermediate deformation state, which may optionally be different from the mechanical behaviour adopted during the calculation of the mechanical equilibrium, to facilitate and accelerate the calculations (or instead, a rest state of the numerical IMD different between the intermediate deformation state and the calculation of the mechanical equilibrium state). Such choices may also prevent the determination of the intermediate deformation state, and/or the calculation of the mechanical equilibrium state, creating local instability modes, thereby adversely affecting the robustness of the simulation faced with the variability of possible IMDs.

It will be noted that the intermediate deformation state is not necessarily calculated in a complex manner by the processing unit using an equation solver. The intermediate deformation state of the IMD fully included in the natural cavity is simpler and faster to calculate than the complete history of deformations of the IMD. In addition, the fact of using a three-dimensional wall model of the natural cavity makes the results much more precise, notably compared to the use of generic nomograms. A customised simulation, for example specific to a patient, is obtained. Preferably, the mechanical behaviour adopted for this natural cavity wall model in the course of the calculation of the mechanical equilibrium state is a non-deformable rigid behaviour, which facilitates the calculation of the mechanical equilibrium state while constituting an acceptable approximation from a physical viewpoint.

The rapid, robust and precise simulation result of the method of the invention enables the health practitioner to conclude rapidly on the predictive efficiency of a given IMD reference for treating the patient, notably in an emergency context. The calculation time from the obtaining of the numerical IMD model may be less than one minute, unlike the average calculation times for methods of the prior art which necessitate the simulation of the real history of deformations. The method of the invention is thus compatible with clinical practice.

Other possible and non-limiting characteristics of the method of the invention, taken alone or in any technically possible combinations thereof, are the following:
a mechanical behaviour of the numerical IMD considered during said determination of the intermediate deformation state is not identical to the mechanical behaviour of the numerical IMD considered during the calculation of the mechanical equilibrium.
a rest state of the numerical IMD considered during said determination of the intermediate deformation state is not identical to the rest state of the numerical IMD considered during the calculation of the mechanical equilibrium.
the mechanical behaviour of the wall model for the calculation of the mechanical equilibrium state is a non-deformable rigid behaviour.
the intermediate deformation state is determined as a function of contact interactions calculated between three-dimensional apexes of the IMD and three-dimensional apexes of the wall model.
during the determination of the intermediate deformation state, the wall model is deformed geometrically from an initial state so as to fully contain the numerical IMD in a rest state of the numerical IMD, the wall model next being brought back to the initial state to obtain the intermediate deformation state of the numerical IMD.
the determination of the intermediate deformation state comprises obtaining a numerical IMD confined in a tool surface associated with an implantation tool model, and integrating, in the wall model, the confined numerical IMD in order to obtain the intermediate deformation state.
the method further comprises a step of determining a central line of the natural cavity, from the wall model, and wherein the numerical IMD is deformed in the course of its integration so as to follow the central line.
the numerical IMD comprises a plurality of segments and further comprises a plurality of nodes, each node connecting the ends of two consecutive segments.
the mechanical behaviour of at least one segment corresponds to the behaviour of a beam, preferably of cylindrical shape.
said segment has a predetermined Young's modulus and/or density and/or Poisson coefficient.
at least one segment of the numerical IMD has a beam mechanical behaviour and is modelled during the determination of the intermediate deformation state with a first diameter, or with a first thickness, and/or with a first elasticity modulus (such as a Young's modulus and/or a Poisson coefficient), and/or with a first slenderness coefficient, and/or with a first gyration radius, and/or with a first set of critical instability loads,
   and said segment is modelled during the calculation of the mechanical equilibrium state respectively with a different second diameter, and/or a different second thickness, and/or a different second elasticity modulus, and/or a different second slenderness coefficient, and/or a different second gyration radius, and/or a different second set of critical instability loads.
the mechanical behaviour of at least one node corresponds to the behaviour of a swivel.
the calculation of the mechanical equilibrium state of the numerical IMD comprises the calculation of a field of displacements Dxi, Dyi, Dzi and a field of rotations Rxi, Ryi, Rzi of each node i of the numerical IMD in a three-dimensional frame of reference linked to the wall model, said two fields being calculated by applying the fundamental dynamic principle on said node.
the calculation of the mechanical equilibrium state of the numerical IMD comprises, for at least one node of the numerical IMD, the calculation of a normal force and/or a friction force applied by the wall model on said node, modelling respectively the penetration resistance of the wall and the friction between the IMD and the wall.
the segments and the nodes of the numerical IMD have at least one end pole, preferably at least two end poles.
a general shape of the IMD is flattened at the level of the end pole, preferably at the level of the end poles.
the end pole(s) is (are) modelled with a first concavity during the determination of the intermediate deformation state, and with a second concavity different from the first concavity during the calculation of the mechanical equilibrium state.
the numerical IMD is a model of an intrasaccular cage.
the numerical IMD is a model of a laser-cut stent.
the method comprises a later step of calculating a predictive apposition of at least one part of the three-dimensional apexes of the numerical IMD on the wall model, preferably of calculating an apposition of a plurality of nodes of the numerical IMD on the wall model.
the numerical IMD corresponds to an IMD reference derived from a set of IMD references recorded in a database,
   steps i., ii. and iii. such as defined above being repeated for each reference of the set of references.
the method comprises a later step of determining a real IMD the most suitable for an implantation in the natural cavity, among the set of references, as a function of the deformed state of the IMD after implantation for each reference of the set of references, and/or as a function of the apposition of said IMD after implantation on the walls of the natural cavity.

According to a second aspect, the invention relates to a computer programme product comprising code instructions for the implementation of the simulation method defined above, when said code instructions are executed by a processing unit.

According to another aspect, the invention relates to a processing unit comprising:

means for obtaining a three-dimensional wall model of a natural cavity,
means for obtaining a numerical IMD, preferably configured to generate the numerical IMD in accordance with an IMD reference derived from a database,
calculation means configured to determine an intermediate deformation state wherein the numerical IMD is deformed as a function of a shape of the wall model, while remaining included in said shape,
the calculation means being further configured to calculate a mechanical equilibrium state of the numerical IMD as a function of a mechanical behaviour of the numerical IMD and a mechanical behaviour of the wall model,
the processing unit being configured to implement a simulation method such as defined above.

### GENERAL DESCRIPTION OF THE FIGURES

Other characteristics, aims and advantages of the invention will become clear from the description that follows, which is purely illustrative and non-limiting, and which should be read with regard to the appended figures among which:

FIG. 1 schematically illustrates an assembly for simulating the deformation of an IMD after implantation, according to an example.

FIG. 2 represents the steps of a method for determining the deformation of an IMD after implantation according to a first embodiment.

FIGS. 3a and 3b represent the successive steps of generating a numerical vascular tree wall model.

FIG. 4 represents a numerical model of IMD of intrasaccular cage type.

FIG. 5 is a schematic diagram of an exemplary modelling possible for an IMD, illustrating a node modelled by a swivel.

FIGS. 6a to 6d represent successive states of an intrasaccular cage model and a wall model in the course of the simulation of post-implantation deformation of the cage according to the method of FIG. 2.

FIG. 7 represents the steps of a method for determining the deformation of an IMD after implantation according to a second embodiment.

FIG. 8 represents a numerical model of IMD of “laser-cut stent” type.

FIGS. 9a to 9f represent successive states of a stent model in the course of the simulation of post-implantation deformation of the stent according to the method of FIG. 7. In FIGS. 9a, 9e and 9f, the stent model is included in the wall model of a natural cavity of a patient. In FIGS. 9b to 9d, the stent model is confined in a micro-catheter represented outside of the natural cavity.

FIG. 10 illustrates a numerical IMD simulating the final shape and the apposition of a “laser-cut” type stent, superimposed on an image of a real stent deployed inside a real vascular tree.

### DETAILED DESCRIPTION OF EMBODIMENTS OF THE INVENTION

Hereafter, reference will indiscriminately be made to an “implant” or an “implantable medical device” (or IMD) to designate an expandable implant, being able to adopt a final position (after implantation and after deployment) within a natural cavity which is different from its initial position (after implantation and before deployment), and which is also different from its position at rest (deployment in the open air).

Such an implant typically has a structure composed of a material that is biocompatible for human tissue. The implant is generally maintained in a compressed position by an implantation tool, for example by a catheter, at the start of the implantation.

Hereafter, an example will be considered where the natural cavity to treat is an artery of a human or animal patient. It will however be understood that the invention may be applied, with the same advantages, to any other body conduit capable of receiving an IMD.

The implantation of an IMD simulated by means of the methods for determining IMD deformation described hereafter is then carried out by endovascular route. The implantation is for example performed by interventional type radioscopic guiding, using an implantation tool such as a micro-catheter.

“Geometric characteristics” or “morphological characteristics” of the natural cavity are taken to mean characteristics of the shape of the cavity, which locally influence the final position of the implant—notably, but not limited to, the minimum diameter, the perimeter, the curvature radii and their spatial derivatives.

Furthermore, a region of interest, comprising the artery to treat, could be designated by the abbreviation “ROI”. The zone of an aneurysm constitutes an example of ROI.

In all of the figures and in the description hereafter similar elements bear identical alphanumerical references.

System for Simulating Deformation of an IMD after Implantation

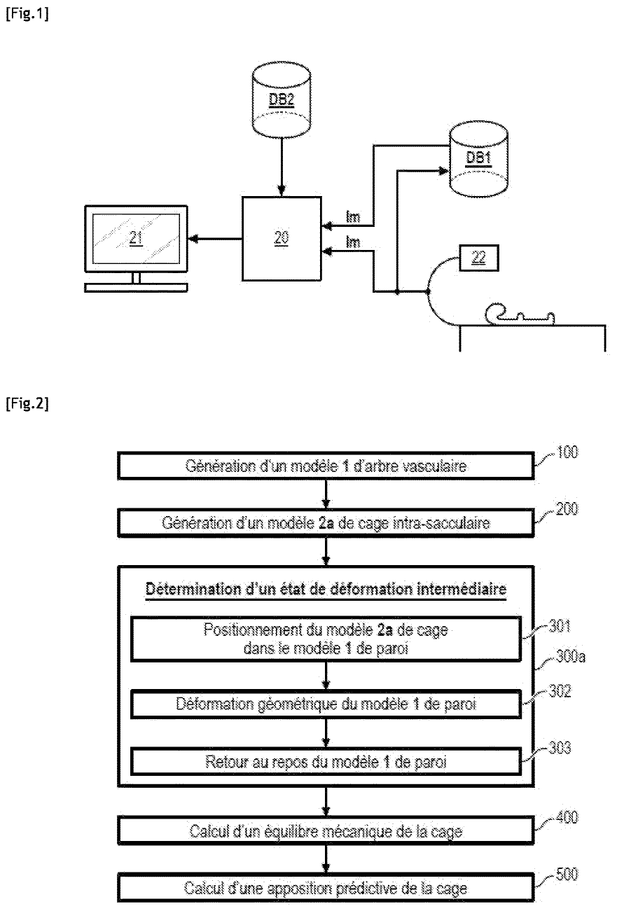

In FIG. 1 is represented a system for determining the positioning of an IMD comprising a processing unit 20 according to the invention. This processing unit is, for example, a processor configured to implement a method for determining the deformation of an IMD, according to any of the exemplary embodiments described hereafter.

The processing unit 20 is, advantageously, configured to communicate with an acquisition unit 22 capable of acquiring views making it possible to reconstitute a three-dimensional image of a region of interest of a patient. The region of interest comprises an artery.

The acquisition unit 22 may for example be an X-ray imaging system, and the views may for example be acquired within the scope of a neuroradiological procedure, for example a three-dimensional angiography acquisition.

The processing unit 20 communicates with the acquisition unit 22 and/or with the database DB1 so as to receive images Im, by wired link and/or by wireless link by means of any suitable network (for example Internet). In an alternative, the processing unit can extract the images from a hard disc, or receive the images from a peripheral storage support reading device such as a CD reader or a USB port.

In an alternative or in combination with the acquisition unit 22, the processing unit 20 is capable of communicating with a database DB1 in which are recorded three-dimensional images of a natural cavity to treat of a patient and/or views making it possible to reconstitute such three-dimensional images.

The processing unit further comprises a database DB2 comprising data concerning implantable medical device (IMD) references. Said data may be supplied by IMD manufacturers, or determined analytically or experimentally. As will be seen hereafter, the processing unit 20 comprises numerical model generation means, capable of generating a numerical IMD in accordance with an IMD reference derived from the database DB2.

The data associated with an IMD reference in the database DB2 comprise physical IMD characteristics such as a maximum diameter and/or a maximum length, and/or pre-recorded mechanical IMD models, for example models in the form of a network of segments connected together by nodes.

According to a possible alternative, the database DB2 comprises a set of IMD references among which a particular reference may be selected to launch the simulation according to the method hereafter. This alternative is advantageous because it enables the user to obtain simulation results for several different IMD references, after which a reference giving the most satisfactory results (as an example, the best apposition on the walls of the cavity) is chosen for the intervention.

In an alternative, the database DB2 is remote from the processing unit 20 and a link between the processing unit 20 and the database DB2 is made by any suitable means, for example wireless means via a communication network.

The processing unit 20 is further connected to a display device 21, providing a graphic interface to a user for the display of three-dimensional images modelling a region of interest typically comprising a natural cavity to treat.

The display device 21 may further be configured to display simulation results derived from the implementation of the method which will be described hereafter. It displays for example three-dimensional views of a numerical IMD after simulation of the deployment in the region of interest. The display device 21 displays a user interface for the input of instructions and, optionally, for the selection of IMD references. The user of the processing unit 20 and the associated display device 21 is for example a health practitioner.

Method for Simulating Deformation of an IMD after Implantation

The system represented in FIG. 1, which comprises in particular the processing unit 20, may be used for the implementation of a method for simulating the deformation of an IMD after its implantation in an artery of a patient. The natural cavity is then a region of interest within a vascular tree of the patient.

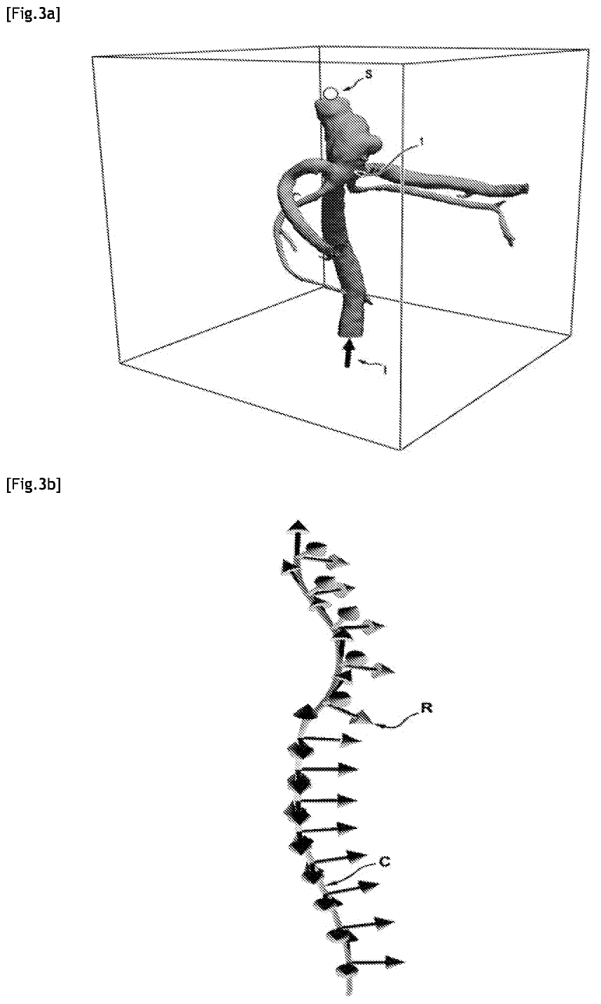

In FIG. 2 is represented an example of simulation method being able to be implemented by the processing unit 20, wherein the IMD to simulate is an intrasaccular cage which must serve for the treatment of an aneurysm at the level of the region of interest. It will be noted that the simulation method according to this example may be used for other types of self-expandable IMDs.

From a three-dimensional model of the artery wall at the level of the ROI, and if needs be from a three-dimensional IMD model (hereafter “numerical IMD”), which corresponds for example to the IMD at rest such as it may be found “off the shelf”, the simulation makes it possible to obtain a three-dimensional model of the IMD in mechanical equilibrium within the artery, after implantation in the artery and after deployment.

Here, the result of the simulation comprises the relative position of the points of the numerical IMD with respect to the points of a wall model representing the ROI of the artery, in a mechanical equilibrium state.

This simulation method is notably useful in the context of a very rapid decision taking by a health practitioner, for the choice of an IMD to implant in a patient who has just suffered or is in the process of suffering a cerebrovascular accident (CVA). The simulation may be used with the same advantages for the treatment of stenoses, thrombectomies, for the replacement of heart valves or for the treatment of aneurysms of the abdominal aorta.

It will be noted that the simulation of deformation after implantation of the IMD may be carried out upstream of this implantation, or during the implantation.

It is crucial to have available a simulation that has a very high level of security and performance to ensure the physical integrity of the patient and the efficacity of the treatment, while being quick enough to enable a decision to be taken in real time. From the moment that a three-dimensional model of the artery wall at the level of the ROI is available, and for a processing unit having a calculation power that is standard in the medical computing field, the totality of the simulation for a given IMD reference may be implemented in a short time, for example between 5 and 60 seconds. The simulation must also be robust and take account of the anatomical specificities of the natural cavity to treat in the patient.

Numerical Wall Model of the Cavity to Treat

At an optional step 100, a model 1 of the artery wall of the patient, encompassing the ROI to treat, is generated by the processing unit 20 or by separate calculation means capable of exchanging data with the processing unit.

Alternatively, the model 1 of the artery wall has been produced prior to the simulation and is obtained by the processing unit 20 from a medical database.

The wall model 1 is typically obtained by image processing, from three-dimensional images of the patient's artery obtained for example by rotational 3D angiography. The three-dimensional image of the artery may be segmented by the “marching cubes” method known in the image reconstruction field.

The three-dimensional images are here extracted directly from the acquisition unit 22. In an alternative, the three-dimensional images may be obtained from the database DB1.

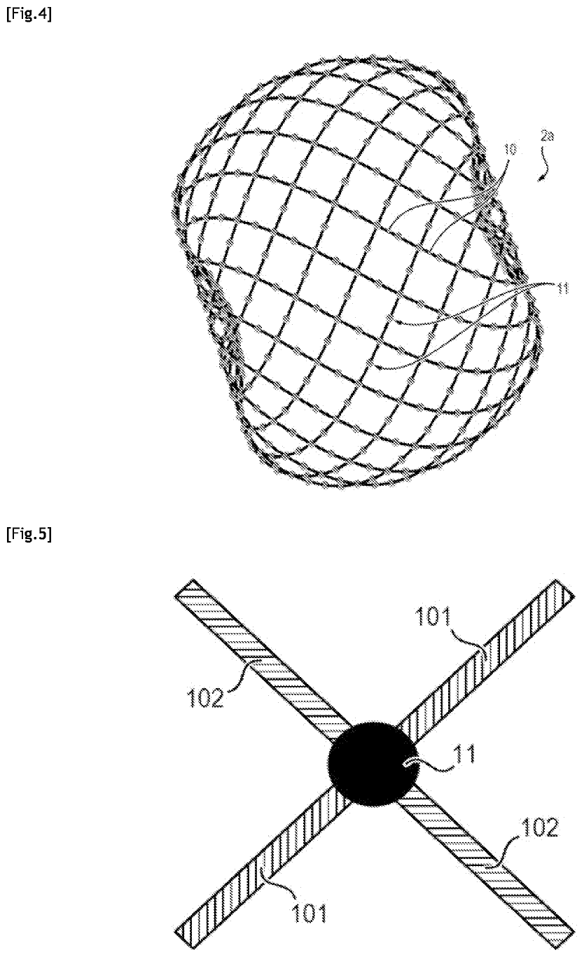

FIG. 3a represents an exemplary artery wall model 1. An entry point I and an exit point S have been defined, for example manually by a practitioner. In an alternative, the entry and exit points are detected automatically. The point I is a point through which an implantation tool, such a micro-catheter, comprising the compressed IMD may be inserted in the ROI during the intervention.

The wall model 1 preferably comprises a discretised three-dimensional surface which approximates the real wall of the artery. The model 1 comprises for example a surface formed of triangles adjacent to one another, said surface being flat inside a given triangle.

Preferably, a central line C of the artery at the level of the ROI is further generated or obtained by the processing unit. In FIG. 3b is represented a central line C calculated from the wall model 1 of FIG. 3a.

The central line C is advantageously oriented. It then comprises a set of points of space. A local basis R, preferably direct orthonormal, is obtained at a plurality of these points, optionally at each of these points. In an alternative, the basis obtained may be non-direct orthonormal.

As an example, the central line C may be calculated by minimising the journey time of fluid particles along the wall model 1. The central line then corresponds to the fastest path so that the particles migrate from the entry point I up to the exit point S. This fastest path may, as an example, be calculated by considering the hypothesis according to which the speed of a fluid particle is proportional to its distance with respect to the vascular wall.

The calculation of the central line C is advantageous for the initial positioning of the numerical IMD. The numerical IMD may be positioned at any point of the central line inside the wall model.

The calculation of the central line C is furthermore useful for the determination of the intermediate deformation state of the IMD, in the case where the latter is simulated inside an implantation tool such as a micro-catheter. This case is described hereafter.

For the later calculation of the intermediate deformation state and for the calculation of the mechanical equilibrium, it is not necessary that the processing unit 20 has at its disposal a physical model of the mechanical behaviour of the elements of the wall model 1. A geometric representation of the surface of the wall model 1 may suffice.

### Numerical IMD Model—Example of an Intrasaccular Cage

At an optional step 200, an IMD model 2, or “numerical IMD”, is generated by the processing unit 20 or by separate calculation means capable of communicating with the processing unit, and is recorded in the memory of the processing unit.

Alternatively, the processing unit 20 extracts the numerical IMD from a database.

The numerical IMD 2 constitutes a physical and geometric modelling of the IMD, which makes it possible to simulate its interaction with the wall of the artery to treat. At this stage, the numerical IMD preferably corresponds to the implant at rest, such as it may be found “off the shelf”. The numerical IMD 2 is recorded in the form of a series of points of which the three-dimensional coordinates are stored in a memory. Connections between the nodes are preferably also recorded in a memory, which makes it possible to reconstitute segments which discretise the mechanical structure of the IMD.

Advantageously, the set of points of the numerical IMD 2 comprises a plurality of segments 10 joined together by nodes 11. Each node connects together two ends of two consecutive segments. A given node may optionally connect more than two segments. The nodes 11 of the numerical IMD are thus interconnected by the segments 10. The assembly formed by the nodes and the segments forms a mesh which constitutes a discretised model of the shape of the IMD.

This model is particularly relevant for modelling IMDs of low thickness.

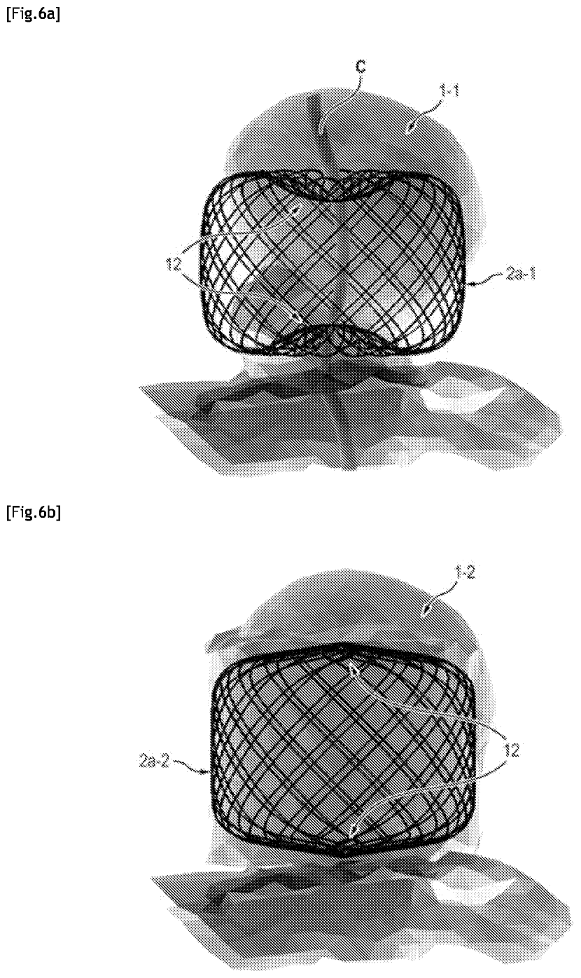

Here, the simulated IMD is an intrasaccular cage composed of a braiding of metal wires made of biocompatible material, interlaced so as to form a grill. A numerical IMD 2a suited to this type of implant, illustrated in FIG. 4, is a set of segments which describe a global spherical shape (flattened at the level of two poles of the numerical IMD).

The numerical IMD obtained or generated by the processing unit may correspond to a reference derived from a set of IMD references recorded in the database DB2. For example, the practitioner can select, via the user interface, a reference corresponding to a shape and/or a particular size of implant at rest, and/or a particular material, and/or a type of implant such as an intrasaccular cage, a laser-cut stent, a flow diverter, an implant of overall conical shape, etc.

For an IMD of intra-aneurysmal cage type, a mechanical model of the braided structure of the IMD in the form of a set of equivalent shells could be adopted. However, the surface of an equivalent shell only varies very slightly in area, and thus does not model in an adequate manner a braided IMD structure. The solutions obtained to simulate the mechanical equilibrium would thus not be very stable.

At this stage, a predetermined mechanical behaviour may be attributed to the elements of the numerical IMD, here to the segments 10 and to the nodes 11 of the numerical IMD 2a.

The mechanical behaviour associated with the elements of the numerical IMD is notably useful for determining the mechanical stresses exerted on the IMD when the latter is in an intermediate deformation state, as will be described hereafter.

It will be noted that the mechanical behaviour of the numerical IMD may not be known during the determination of the intermediate deformation state of the numerical IMD and of the wall model.

The mechanical behaviour of the IMD is, on the other hand, known during the later resolution of the mechanical equilibrium.

As an example, each segment 10 is here considered as a beam element which makes it possible to discretise a neutral fibre of the IMD.

“Neutral fibre” is taken to mean the curve linking the centres of gravity of the straight sections forming the structure of the IMD.

The structure of the IMD thus equates with a tubular volume described by the segments placed end to end along the neutral fibre. The tubular volume is generated by the set of straight sections. The straight sections are here circular, but a modelling with other types of sections (triangular, rectangular, etc.) may be adopted.

This model makes it possible, during the later numerical simulation of the mechanical interactions between the IMD and the wall, to differ at the level of the neutral fibre the forces applied on the tubular volume of the segment.

A set of predetermined parameters may be associated with each beam element (each segment), among which a predetermined Young's modulus E, a Poisson coefficient v and a density p. To simplify the modelling, all the beam elements may be associated with the same parameters.

It is preferably considered that the material constituting these beam elements is elastic, homogeneous and isotropic.

In the specific example of a braided type stent, such as the intrasaccular cage modelled by the numerical IMD 2a, it is preferable, in order to simplify and accelerate the later calculations of the mechanical equilibrium, to ignore relative translational movements between two superimposed wires of the mesh, and the friction caused by the movement of one wire against another.

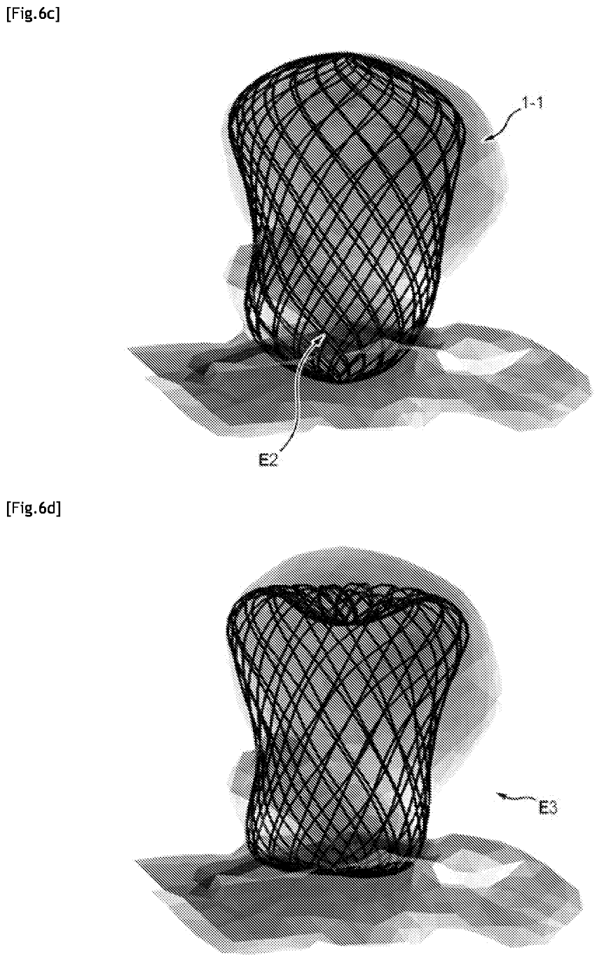

In this respect, it is advantageous to model the nodes situated at the intersection of several wires of the mesh of the intrasaccular cage as a simple, swivel type mechanical link. FIG. 5 is an illustrative diagram of this modelling for a node 11 of the numerical IMD 2a. Here, four segments intersect at the level of the node 11, of which two segments 101 which model a first wire of the cage and two segments 102 which model a second wire of the cage.

The node having a swivel mechanical behaviour, the hypothesis is made that each of the segments is rotationally free in space compared to the other segments (ignoring friction). The segments are not on the other hand translationally free with respect to one another (relative translation of two wires ignored).

This modelling is relevant in the case of a stent having a dense braiding of wires, such as an intrasaccular cage. It reinforces the rapidity, the stability and the robustness of the calculation of deformations applied to the numerical IMD 2a.

It will be noted that the modelling of an IMD as a set of nodes and segments, and the modelling of nodes as swivels, may be used even for a simulation of the deformation of an IMD which would not include the determination of an intermediate deformation state before the resolution of the mechanical equilibrium.

### Determination of an Intermediate Deformation State

Returning to the method of FIG. 2, the simulation next comprises the determination 300a of an intermediate deformation state for the system formed by the wall model 1 and the numerical IMD 2a.

This intermediate deformation state is a theoretical state of the IMD with respect to the wall of the artery. In this intermediate deformation state, the numerical IMD is wholly included inside the wall model.

In the intermediate deformation state of the numerical IMD, the wall model preferably has the same shape as at rest. “Shape of the wall model” is taken to mean the positions in space of the points of the wall model, with respect to one another.

Similarly, the shape of the numerical IMD here depends on the positions of the three-dimensional apexes of the numerical IMD. The shape of the numerical IMD is drawn by the surface joining the nodes.

The wall of the natural cavity is here considered as rigid and non-deformable, notably in the course of the calculation of the mechanical equilibrium between the numerical IMD and said wall. Thus, the calculation of the mechanical equilibrium is robust and rapid, while remaining an acceptable approximation of the reality.

On the other hand, the numerical IMD is not considered as non-deformable. It is deformed as a function of the shape of the wall model.

However, in the method according to the example illustrated in FIG. 2, it is authorised to deform temporarily the wall model to aid the calculation of the intermediate deformation state of the numerical IMD. This momentary deformation of the wall model, during the calculation of the intermediate deformation, is not obligatory.

More specifically, during the calculation of the intermediate deformation state, the numerical IMD 2a is firstly placed, at sub-step 301, in the frame of reference linked to the wall model 1. If a central line C has been calculated, a centre of the numerical IMD 2a is placed on the central line C.

Advantageously, the numerical IMD 2a may be placed at the level of a positioning point (for example an entry point, not illustrated) in the frame of reference linked to the wall model.

The numerical IMD 2a models an intrasaccular cage at rest, in a state where it is not subjected to mechanical stress tending to retract it. For a correct apposition of the cage on the walls of the aneurysm, the cage must be more extended at rest than inside the aneurysm. Thus, the numerical IMD 2a is preferably chosen with a sufficient size to intersect, at rest, the surface of the wall model 1 when said IMD is placed on the central line.

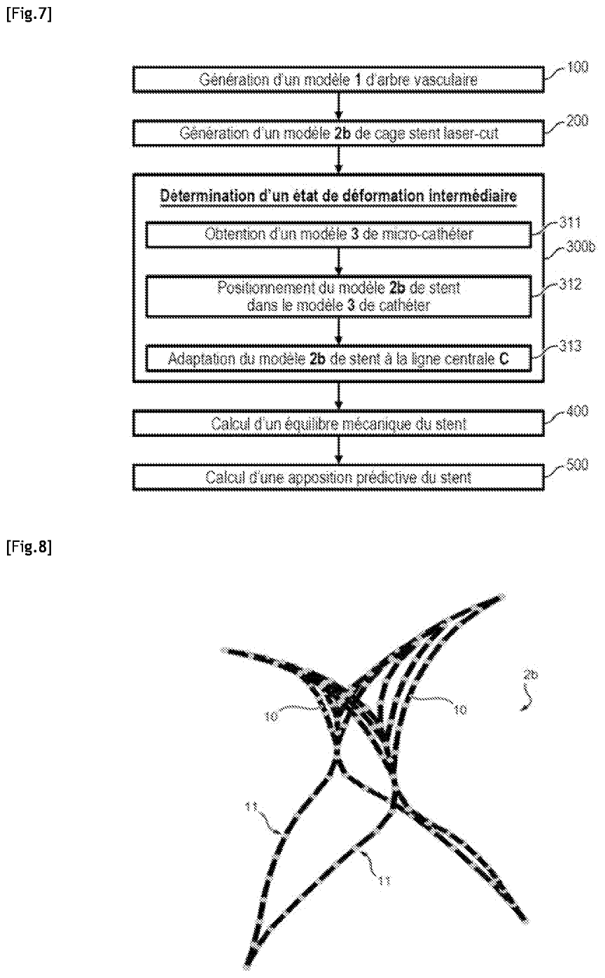

FIG. 6a illustrates the numerical IMD in a first extended state 2a-1 and the wall model in its state 1-1 at rest, at the end of sub-step 301. The reference 12 is attributed to the two opposite end poles of the IMD, which are initially retracted inwards.

The wall model is next enlarged at a sub-step 302, such that the wall model covers the totality of the surface of the numerical IMD. This enlargement corresponds to the momentary deformation of the aforementioned wall model. The deformation of the wall model is here a sub-step of calculating the intermediate deformation state of the IMD inside the wall, but this deformation of the wall model is not conserved for the subsequent calculations of the mechanical equilibrium of the IMD.

At the end of sub-step 302, the nodes 11 of the numerical IMD are surrounded by the surface of the wall model.

To enlarge the wall model, a cylindrical deformation is for example applied to the surface of the wall model 1, as a function of a maximum diameter and/or a maximum height of the numerical IMD 2a at rest.

FIG. 6b illustrates the numerical IMD in a second extended state 2a-2 and the wall model in its enlarged state 1-2 so as to encompass the extended IMD, at the end of sub-step 302.

In the present example, the end poles 12 of the numerical IMD are, in the second extended state 2a-2, reoriented outwards. Thus, the numerical IMD in state 2a-2 is globally convex.

The rest state of the numerical IMD, taken as the starting position of the numerical IMD before considering contact interactions with the wall model, may be selected so as to simplify the calculation of the intermediate deformation state. Notably, the geometry at rest of the numerical IMD (drawn by the segments and the nodes) may be modified between the determination of an intermediate deformation state and the calculation of a mechanical equilibrium state.

In the present example, the geometry at rest of the numerical IMD is modified by reversing the concavity of the end poles of the intrasaccular cage to obtain the second extended state 2a-2, as is visible in FIG. 6b. This geometry has the advantage of making it possible to obtain, in the course of the subsequent calculation of the mechanical equilibrium, a history of deformations closer to the real history of deformations of the IMD.

Returning to FIG. 2, the wall model is next deformed progressively at a sub-step 303 to be brought back to its rest state 1-1 (the wall being considered as rigid and non-deformable), leading to a deformation of the numerical IMD until reaching the sought after intermediate deformation state.

During the progressive return of the wall model to rest, a mechanical calculation of the contact interactions between the IMD and the wall is preferably carried out in an iterative manner. Successive deformations of the numerical IMD are calculated in the course of these iterations, as a function of the contact interactions obtained. At each iteration, the positions of the three-dimensional apexes of the numerical IMD are recalculated. In the course of these successive deformations, the numerical IMD remains included in the wall model.

Preferentially, for the calculation of contact interactions, the mechanical behaviour of the mechanical elements of the numerical IMD—here the nodes and the segments of the intrasaccular cage—is simplified compared to the mechanical behaviour that is considered hereafter for the calculation of the mechanical equilibrium.

In the present example, the thickness of the segments is multiplied by ten to avoid buckling phenomena during step 300a of calculating the intermediate deformation state.

In an alternative or in combination, one or more of the following parameters of segments (these segments each being for example modelled as a beam) could be modified uniquely for step 300a of calculating the intermediate deformation state, notably making it possible to facilitate the calculations: the diameter and/or the thickness and/or an elasticity modulus (Poisson coefficient and/or Young's modulus) and/or the slenderness coefficient and/or the gyration radius and/or one or more critical instability loads.

It will be noted that such choices for modelling the numerical IMD to determine the intermediate deformation state may also be adopted for a numerical IMD of laser-cut stent type (step 300b described hereafter), or of another type.

As an illustrative example, for an IMD of laser-cut stent type, the average diameter of the stent in the intermediate deformation state (for example, stent inserted in a tool surface) may be chosen strictly less than the “real” diameter of the stent calculated during the determination of the mechanical equilibrium state of the IMD.

At the end of the return of the wall to rest at step 303, the numerical IMD has a deformed state which is selected as intermediate deformation state E2. The intermediate deformation state E2 for the present example is illustrated in FIG. 6c.

An advantage of the determination of such an intermediate deformation state is to shrewdly initialise the subsequent calculation of the mechanical equilibrium between the numerical IMD and the wall model.

From the intermediate deformation state, the numerical IMD is going to be progressively relaxed during the calculation of the mechanical equilibrium of the IMD—while taking account this time of a more complex mechanical behaviour for the IMD and optionally for the wall.

By using an intermediate deformation state (theoretical) of the IMD during the subsequent calculation of the mechanical equilibrium, it is not necessary to re-trace the whole of the history of deformation of the IMD between a rest state and a final state. The overall calculating time of the simulation of deployment of the IMD is reduced thereby. The calculation of the mechanical equilibrium remains however reliable and robust, the intermediate deformation state of the numerical IMD being determined as a function of the geometry at rest of the IMD and the shape of the wall of the natural cavity.

### Modelling of the Mechanical Interaction Between the IMD and the Wall

With a view to the calculation of the mechanical equilibrium, it is advisable to model the mechanical interactions between the surface of the IMD and the wall of the cavity. These are mainly the contact interactions which determine the way in which the IMD deforms to adapt to the anatomy of the artery after its implantation.

Advantageously, the wall of the cavity is considered as rigid and non-deformable. The expansion of this wall under the effect of the spontaneous expansion of the IMD after implantation is thus ignored.

Thus, the numerical resolution of the mechanical equilibrium may be carried out from equations of displacement and/or rotation of the points of the numerical IMD in the referential of the wall model.

In the present example, a co-rotational formulation is used to explain the mechanical equations of the displacements and rotations of the nodes 11 of the numerical IMD. The nodes being indexed by the index i, a field of displacements (Dxi, Dyi, Dzi) and a field of rotations (Rxi, Ryi, Rzi) of each node i of the numerical IMD are calculated in a frame of reference linked to the wall model, by applying the fundamental dynamic principle on said node.

Preferably, the inertia of the IMD is ignored and acceleration is taken as zero during the application of the fundamental dynamic principle. The fundamental static principle is thus applied.

To formulate the equations of the fields of displacements and rotations of the nodes, it is advisable to model the forces applied by the wall of the cavity on each node i.

In this respect, a penalisation method is advantageously used.

For each node i, starting from a given state of the numerical IMD (for example the second extended state 2a-2 of the numerical IMD), the processing unit 20 firstly determines if there is penetration of the wall of the cavity by the node i.

During the progressive narrowing of the wall model 1 to determine the intermediate deformation state of the IMD, certain nodes of the numerical IMD re-enter into contact with the surface of the wall model.

Similarly, in the course of the relaxation of the mechanical stresses of the IMD up to determining the mechanical equilibrium between the IMD and the wall, nodes of the numerical IMD re-enter into contact with the surface of the wall model.

If a penetration at the level of a node i is detected, the forces exerted on the node i are modelled by a normal force Fₙₒᵣₘₐₗ and a friction force F_{friction} applied by the wall model on said node, modelling respectively the resistance of the wall to penetration of the node, and the friction between the IMD and the wall.

The norm of the force Fₙₒᵣₘₐₗ is taken equal to the product k×p, with k the rigidity of a spring modelling the contact rigidity of the wall and p the distance of penetration of the node i in the wall model, according to a direction normal to the wall. The force Fₙₒᵣₘₐₗ is all the greater when the penetration and the rigidity of the spring are high.

The force F_{friction} is modelled in a direction tangential to the wall, and its norm is taken equal to the norm of the force Fₙₒᵣₘₐₗ multiplied by a friction coefficient μ.

In an alternative, the model of the mechanical interactions could integrate only the force Fₙₒᵣₘₐₗ, which corresponds to a contact without friction. However, it is preferable to integrate the friction force and the normal force to guarantee good precision of the simulation of mechanical equilibrium.

In addition, taking friction into account accelerates the convergence of the calculation of the mechanical equilibrium state and thus reduces the simulation times.

The penalisation method used above to obtain the mechanical equations of displacements and rotations provides a good compromise between rapidity of calculation, robustness of the mechanical model and precision of the simulation results. It will be noted that this penalisation method can also be used with a numerical IMD modelled other than by means of nodes and segments.

It will be understood that other numerical methods may be envisaged, in combination or as a replacement of the penalisation method, for the calculation of the contact interactions between the IMD and the wall.

Preferentially, boundary conditions may be imposed on certain apexes of the numerical IMD and taken into consideration in the mechanical behaviour of the IMD.

The translational and/or rotational displacements of certain nodes of the numerical IMD, for example nodes situated on a lower edge of the numerical IMD, may be constrained during the calculation of successive deformations of the IMD. An advantage of using boundary conditions at the level of the apexes of an inner edge of the IMD is to improve the modelling of the contact at the level of the attachment of the IMD with an implantation tool, such as a micro-catheter.

These boundary conditions are used during the calculation of the intermediate deformation state and/or during the calculation of the mechanical equilibrium of the numerical IMD.

A first advantage of the use of boundary conditions is to make the system of equations to resolve better conditioned, and to make the solutions obtained more stable.

A second advantage, involving in particular the determination of the mechanical equilibrium of the numerical IMD, is to guide the progressive deformation of the numerical

IMD to a final deformation state closer to clinical reality.

### Calculation of the Mechanical Equilibrium

From the intermediate deformation state E2 obtained previously, a mechanical equilibrium state E3 of the numerical IMD is calculated by obtaining mechanical stresses at the level of the apexes of the numerical IMD, and by simulating the relaxation of these stresses by calculation.

The relaxation of the mechanical stresses exerted on the apexes of the IMD corresponds to an iterative calculation of successive deformation states of the numerical IMD, until reaching a position considered as a position of mechanical equilibrium with the wall model.

In the course of these successive states, the numerical IMD 2 is progressively relaxed to conform to the shape of the wall model 1, just like the real behaviour of the IMD tending towards its rest position while deploying inside the artery.

The stresses obtained by calculation depend on the respective mechanical behaviours of the numerical IMD and the wall model. For the calculation of the mechanical equilibrium, the mechanical stresses exerted on the IMD by the wall are introduced. The mechanical stresses here comprise the contact interactions between the IMD and the wall, calculated for example according to the modelling defined above.

It will be recalled that the behaviour of the wall model 1 during the calculation of the mechanical equilibrium is preferentially chosen as rigid non-deformable. Thus, the wall has the same shape during the mechanical equilibrium calculation as at the start of the simulation—except that, as indicated above, it has been possible to deform momentarily the wall model in the course of the determination of the intermediate deformation state.

Returning to FIG. 2, the calculation of the deformation of the IMD to reach mechanical equilibrium is carried out a step 400 by resolution of equations representative of the mechanical interactions between the mechanical elements of the numerical IMD and the wall model.

The mechanical equilibrium state is optionally the final of several progressive deformation states calculated from the intermediate deformation state.

The formulation used for the equations of the mechanical interactions between the IMD and the wall preferentially corresponds to the co-rotational formulation of the fields of displacements and rotations of the nodes of the numerical IMD 2, such as defined above.

FIG. 6d illustrates the numerical IMD and the wall model of FIGS. 6a to 6c at the end of the mechanical equilibrium calculation. From the state of FIG. 6c, the mechanical equilibrium state E3 of the numerical IMD is calculated by considering the real geometry of the IMD in the rest state.

It will be recalled that, in the extended state 2a-1, the end poles 12 of the IMD are retracted inwards. In the course of the mechanical equilibrium calculation, the mechanical stresses exerted on the DMI are relaxed and, due to this relaxation of the stresses, the end poles 12 retract again spontaneously inwards. Thus, the concavity of the end poles 12 is chosen different between the intermediate deformation state of the numerical IMD (here the intrasaccular cage) and its mechanical equilibrium state.

The mechanical equilibrium calculation carried out here is of non-linear type. It is possible to obtain an approximation of the mechanical interactions that is very close to reality. The prediction of the shape and the final disposition of the implant in the natural cavity is thus very reliable and precise.

In this example, the mechanical equilibrium is calculated for each of the nodes of the numerical IMD 2a. In an alternative, the resolution may only be carried out for certain points, the positions of the other nodes at equilibrium then being extrapolated from those of these points.

The calculation of the mechanical equilibrium is considered as finished when a criterion of convergence of the deformation states, recorded in the processing unit, is reached.

The calculation of the mechanical equilibrium from the intermediate deformation state improves the efficiency of the simulation, due to the rapidity and the stability of the solutions obtained.

The taking into account of all the interactions between the IMD, the wall of the natural cavity and a micro-catheter would necessitate modelling the longitudinal or “push/pull” compression applied by the practitioner via the implantation device, during the placement of the IMD. This model proves to be slow and not very robust in practice. Such a modelling thus cannot be used in certain situations requiring rapid decision taking, for example in less than one minute, from obtaining three-dimensional images of the vascular wall.

### Alternative Example—Simulation of Deformation of a Laser-Cut Stent

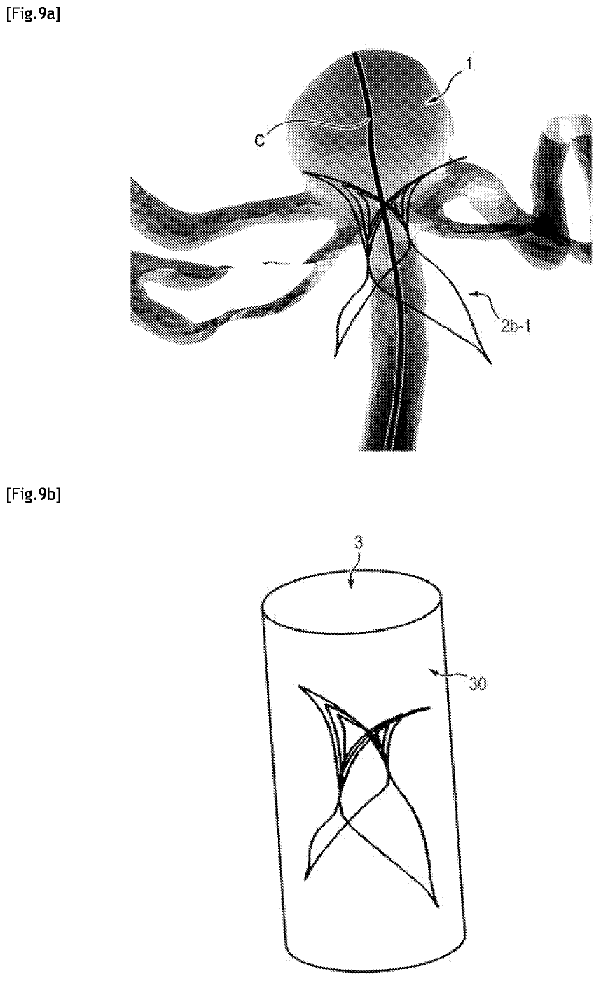

In FIG. 7 is represented a simulation method being able to be implemented by the processing unit 20, according to a second example distinct from that of FIG. 2.

The IMD to simulate is of “laser-cut stent” type in the present example. This example of IMD does not have an overall spherical shape. It will be noted that the simulation method according to this second example may be used for all types of expandable IMD, even if its use for an IMD of laser-cut stent type is described here.

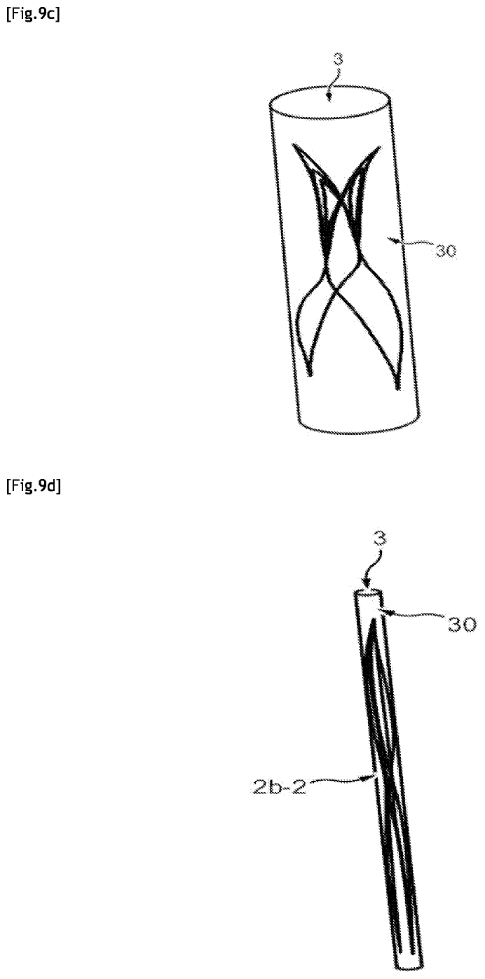

FIG. 8 illustrates a three-dimensional view of a numerical IMD 2b constituted of segments 10 and nodes 11 generated to simulate the shape of the laser-cut stent. Preferably, the behaviour attributed to the mechanical elements of this model is similar to that of the IMD 2a of the above example, with the exception of the fact that it is not necessary to model the intersection between the segments (the wires) of the numerical IMD by swivels. Indeed, the laser-cut type stent is not a braided implant, and the swivel model is thus less relevant here.

A compressed state in an implantation tool and a mechanical equilibrium state in the artery, the longitudinal deformation is made to be more important than for the first exemplary simulation method described above in relation to FIG. 2.

Returning to the method of FIG. 7, after obtaining a model 1 of the wall of the natural cavity (here the artery) and a model 2b of the stent of which the deformation is simulated, the processing unit implements a determination 300b of an intermediate deformation state for the system formed by the wall model 1 and the numerical IMD 2a.

In the same way as in the preceding example, the sought after intermediate deformation state is a theoretical state of the IMD with respect to the wall of the artery, wherein the numerical IMD is wholly included inside the wall model.

This intermediate deformation state is theoretical, and does not necessarily involve calculating the mechanical interactions between the IMD and the wall.

The intermediate deformation state is here obtained by the sub-steps of:
positioning the numerical IMD in the rest state inside the wall model, along the central line,
obtaining 311 a model of an implantation tool 3, here a micro-catheter, the tool model notably comprising a tool surface 30,
generating 312 the numerical IMD 2b confined in the tool surface 30,
positioning 313 in the wall model 1 the numerical IMD 2b in the confined state, so as to obtain the intermediate deformation state E2. The tool model is not, for its part, positioned inside the wall model 1.

The simulation next continues, in a manner similar to the first exemplary simulation method described in relation to FIG. 2, by a deformation of the numerical IMD to the intermediate deformation state, then a relaxation of the mechanical stresses undergone by the numerical IMD until reaching a mechanical equilibrium state.

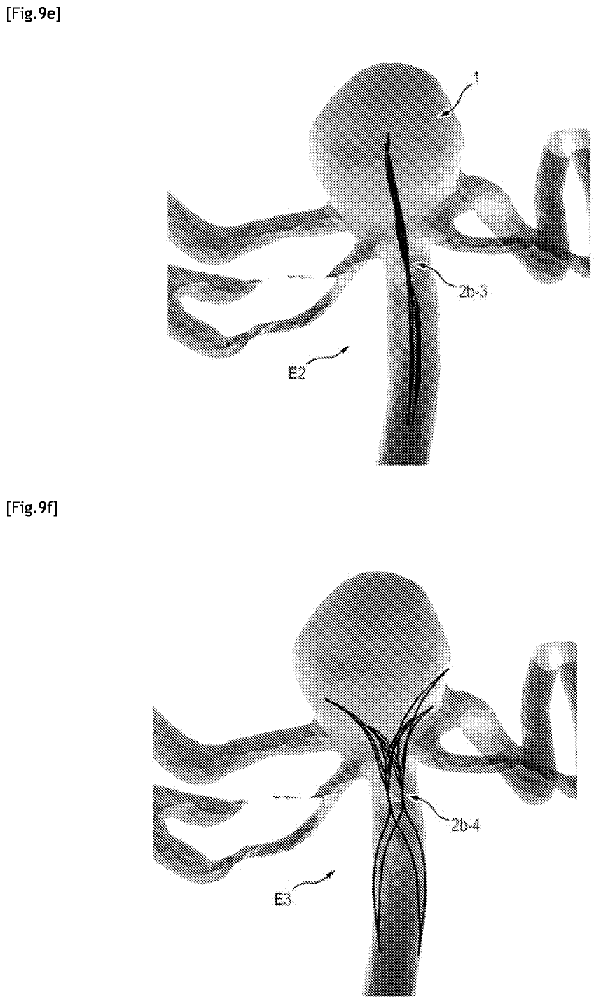

FIGS. 9a to 9e illustrate the obtaining of the intermediate deformation state.

In FIG. 9a, after obtaining beforehand the central line C of the artery, the numerical IMD in its state 2b-1 is positioned in the neighbourhood of a zone to treat, for example at a positioning point situated along the central line C.

The state 2b-1 corresponds to the IMD at rest, without stress. The numerical IMD then intersects the wall model at a plurality of zones, the mechanical interactions between the IMD and the wall not being taken into account.

At sub-step 311, a micro-catheter, of length preferentially substantially greater than that of the numerical IMD in the state 2b-1, is generated in an initial state. A length of catheter greater than that of the numerical IMD is preferable, because the IMD is made to lengthen all the more since it will be compressed in a small diameter of micro-catheter.

The simulated micro-catheter is for example of cylindrical shape. Its radius is preferably smaller than the minimum radius of the natural cavity in the ROI.

It will be noted that the implantation tool model is not necessarily generated by the processing unit 20, but may be recovered in a database.

In the course of a sub-step 312, the numerical IMD is inserted in the model of implantation tool (here a micro-catheter). The surface 30 of the micro-catheter is, firstly, dilated such that the micro-catheter encompasses the numerical IMD in the rest state 2b-1. The micro-catheter thus encompassing the numerical IMD is represented in FIG. 9b (separately from the wall model of the natural cavity of the patient).

Next, the surface 30 is progressively retracted to bring the micro-catheter back to its initial state, the numerical IMD remaining included in the micro-catheter.

In the course of the progressive retractation of the surface of the micro-catheter, the contact interaction between the IMD and the surface of the micro-catheter is resolved. The IMD is thus compressed progressively, while passing through an intermediate position illustrated in FIG. 9c, until reaching the deformation state 2b-2. At the end of the successive calculations, a confined state 2b-2 of the IMD is obtained, represented in FIG. 9d.

The numerical IMD in the confined state 2b-2 is compressed in the tool surface 30 of the micro-catheter. The confined numerical IMD 2b-2 is ready to be included in the wall model. The micro-catheter generated previously is not on the other hand included inside the wall model.

In an advantageous alternative, the sub-steps of calculating the confined state of the IMD are only implemented once for each IMD reference, “offline” upstream of the simulation. The confined state of the IMD is stored in a database, to be reused later during the simulation and to determine the intermediate deformation state of the numerical IMD in a natural cavity.

An advantage of a calculation of the confined state of the IMD carried out “offline” is to greatly reduce the simulation time, which increases the reactivity of the simulation and accelerates the potential choice of the IMD reference to implant.

In the case of an “offline” calculation, sub-step 312, implemented for the simulation consists simply in recovering in the database the numerical IMD in the confined state in the tool surface.

Next, at sub-step 313, the numerical IMD is integrated inside the wall model 1, in the referential of the wall model.

Advantageously, the numerical IMD 2b is deformed in the course of its positioning at step 313, from its compressed state 2b-2 in the tool surface 30, so as to make it follow the central line C of the artery provided with a curvilinear abscissa and its local frame of reference.

The numerical IMD thus reaches a state of deformation 2b-3.

By inserting along the central line the numerical IMD in its state 2b-3, an intermediate deformation state E2 of the numerical IMD is obtained, wherein the numerical IMD is totally included in the wall. Such an intermediate deformation state E2 is represented in FIG. 9e.

Advantageously, for the alignment of the numerical IMD along the central line and to obtain the intermediate deformation state, a mechanical behaviour of the elements of the numerical IMD is not taken into account. The transformation of the numerical IMD is then uniquely geometric at this stage.

Finally, from the intermediate deformation state E2, a mechanical equilibrium state E3 between the numerical IMD 2b and the wall model 1 may be calculated by the processing unit 20, in a robust and rapid manner, at a step 400.

Successive deformation states, taking account of the mechanical interactions between the numerical IMD and the wall model, are calculated in an iterative manner up to convergence towards mechanical equilibrium. Here, a plurality of portions of the numerical IMD are deformed in the local orthonormal frame of reference R of the central line C. The portions of the IMD are for example successive longitudinal portions along the central line.

The methods described previously in relation to the method of FIG. 2 are advantageously used for this purpose: penalisation method for the modelling of the mechanical interactions between the IMD and the wall and/or co-rotational formulation of the fields of deformations and rotations on the nodes of the numerical IMD, etc. Other methods for numerically resolving the contact interactions between IMD and wall could however be used.

In FIG. 9f is illustrated the numerical IMD in a state 2b-4 in which mechanical equilibrium is reached. The mechanical equilibrium state corresponds to the simulated deformation of the IMD after its implantation in the natural cavity of the patient.

### Calculation of the Predictive Apposition of the IMD on the Wall

From the mechanical equilibrium state E3 obtained for the wall model 1 and for the numerical IMD 2, it is advantageous to calculate the distance between points of the numerical IMD and the surface of the wall. Notably, if a modelling of nodes and segments is adopted for the numerical IMD, this distance is obtained for a plurality of the nodes of the numerical IMD, or even for all of these nodes.

In this respect, the simulation methods illustrated respectively in FIG. 2 and in FIG. 7 comprise a step of calculating a predictive local apposition of the IMD, comprising the calculation of the distance between the nodes of the numerical IMD and the wall model at the equilibrium state E3 obtained above.

The distance data thus calculated are advantageously illustrated by a map representative of the predictive local apposition of the implant against the wall of the natural cavity. One speaks of “local” apposition because this apposition is specific to each apex of the numerical IMD.

For example, from a three-dimensional image of the wall model 1 and the numerical IMD 2 in the mechanical equilibrium state E3, different colours are associated with the zones of the numerical IMD, as a function of the apposition of the nodes contained in these zones, to obtain the apposition map. The colour green is associated with zones of the IMD considered as correctly apposed, and the colour red is used for zones having incorrect apposition.

A threshold distance may be pre-recorded in a memory of the processing unit. A point of the numerical IMD (for example a node) of which the distance to the surface of the wall model is below this threshold distance is considered as being in contact with the wall model, which corresponds to correct apposition. It will be understood that the distinction between correct and incorrect apposition, and thus the coloration of the zones of the IMD, depends on the pre-recorded or selected threshold distance.

Advantageously, the predictive local apposition map of the IMD is displayed on the graphic interface provided by the display device 21. The practitioner can choose an IMD reference to implant, or confirm the choice of IMD made, by noting the quality of the apposition of the IMD against the natural cavity at the level of the ROI.

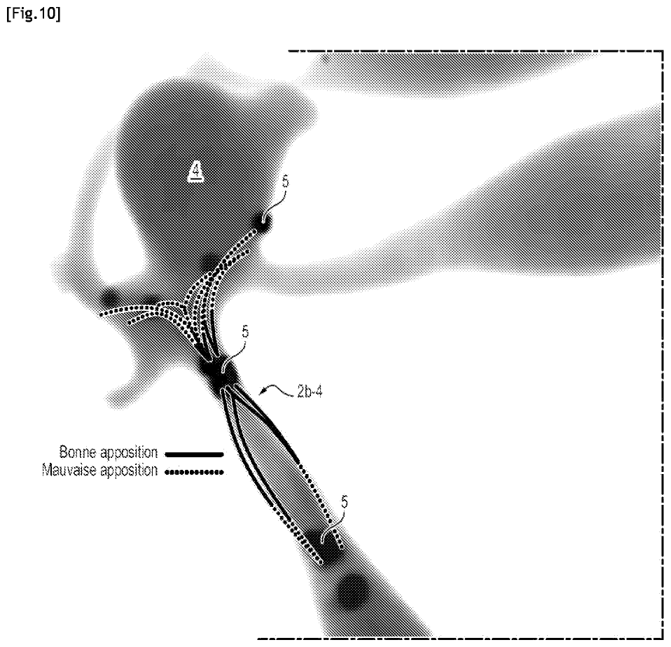

In FIG. 10 is thus represented an exemplary apposition map resulting from the simulation of deformation of an IMD of “laser-cut stent” type. A good apposition for the central zone of the IMD is observed. Zones of the IMD close to the ends have poorer apposition; for such a stent, it is not expected that all the surface of the IMD is apposed against the walls of the artery.

The shape of the numerical IMD 2b-4 at mechanical equilibrium has here been obtained in accordance with the method described above in relation with FIG. 7.

FIG. 10 also comprises, in superposition with the apposition map associated with the numerical IMD, a view of the real IMD 5 after expansion within the artery 4. This latter view stems from 3DRA images of the artery.

It will be noted that the simulation result, corresponding to the numerical IMD 2b-4 of FIG. 10, is obtained in only 6 seconds. The simulation method is thus very rapid and robust.

Furthermore, the results of simulation of deformation of the IMD after implantation are very close to clinical reality, the ends of the model being close to the points of the real IMD 5 visible in 3DRA images. The simulation method is very precise.

From the predictive apposition data for the IMD after deformation on a plurality of points of the numerical IMD, an average predictive apposition can be calculated. In the case where several IMD references derived from a set of references have been simulated, the simulation makes it possible to determine the IMD reference for which the average predictive apposition is the highest. The practitioner can use this information to finalise his most suitable IMD reference.

However, the practitioner can make his choice as a function of other information resulting from the simulation of deformation of the IMD after implantation. For example, the practitioner can dismiss IMD references for which an undesired obstruction of arteries neighbouring the zone to treat is predicted.

1. Method for simulating the deformation, after implantation, of an implantable medical device, called IMD, in a natural cavity, from a three-dimensional numerical model of a wall of the cavity,
the method comprising the following steps implemented by a processing unit:
i. determining an intermediate deformation state of a numerical IMD representing the IMD, the numerical IMD at the intermediate deformation state being deformed as a function of a shape of the wall model while remaining included in said shape,
ii. calculating of a mechanical equilibrium state of the numerical IMD from the intermediate deformation state, the step of calculating comprising calculating mechanical stresses undergone by the numerical IMD in the intermediate deformation state which are a function of a mechanical behaviour of the numerical IMD and a mechanical behaviour of the three-dimensional numerical model of a wall of the cavity, and also comprising the relaxation of said stresses,
wherein a mechanical behaviour of the numerical IMD, and/or a rest state of the numerical IMD, during the step determining the intermediate deformation state is not identical respectively to the mechanical behaviour of the numerical IMD, and/or to a rest state of the numerical IMD, during step of calculating the mechanical equilibrium, wherein the calculated mechanical equilibrium state corresponds to the simulated deformation of the IMD after implantation.

2. Method according to claim 1, wherein the intermediate deformation state is determined as a function of contact interactions calculated between three-dimensional apexes of the IMD and three-dimensional apexes of the wall model.

3. Method according to claim 1, wherein the mechanical behaviour of the wall model for the calculation of the mechanical equilibrium state is a non-deformable rigid behaviour.

4. Method according to claim 1, wherein, during the determination of the intermediate deformation state, the wall model is deformed geometrically from an initial state so as to contain wholly the numerical IMD in a rest state of the numerical IMD, the wall model next being brought back to the initial state to obtain the intermediate deformation state of the numerical IMD.

5. Method according to claim 1, wherein the determination of the intermediate deformation state comprises:
obtaining a numerical IMD confined in a tool surface associated with a model of implantation tool,
integrating in the wall model, the confined numerical IMD in order to obtain the intermediate deformation state.

6. Method according to claim 5, further comprising a step of determining a central line of the natural cavity, from the wall model, and wherein the numerical IMD is deformed in the course of its integration so as to follow the central line.

7. Method according to claim 1, wherein the numerical IMD comprises a plurality of segments and further comprises a plurality of nodes, each node connecting the ends of two consecutive segments.

8. Method according to claim 7, wherein the mechanical behaviour of at least one segment corresponds to the behaviour of a beam, preferably of cylindrical shape.

9. Method according to claim 8, wherein at least one segment of the numerical IMD having a beam mechanical behaviour is modelled during the determination of the intermediate deformation state with a first diameter, and/or with a first thickness, and/or with a first elasticity modulus, and/or with a first slenderness coefficient, and/or with a first gyration radius, and/or with a first set of critical instability loads,
and wherein said segment is modelled during the calculation of the mechanical equilibrium state respectively with a different second diameter, and/or a different second thickness, and/or a different second elasticity modulus, and/or a different second slenderness coefficient, and/or a different second gyration radius, and/or a different second set of critical instability loads.

10. Method according to claim 8, wherein the mechanical behaviour of at least one node corresponds to the behaviour of a swivel.

11. Method according to claim 8, wherein the calculation of the mechanical equilibrium state of the numerical IMD comprises the calculation of a field of displacements Dxi, Dyi, Dzi and a field of rotations Rxi, Ryi, Rzi of each node i of the numerical IMD in a three-dimensional frame of reference linked to the wall model, said two fields being calculated by applying the fundamental dynamic principle on said node.

12. Method according to claim 8, wherein the calculation of the mechanical equilibrium state of the numerical IMD comprises, for at least one node of the numerical IMD, the calculation of a normal force and/or a friction force applied by the wall model on said node, modelling respectively the penetration resistance of the wall and the friction between the IMD and the wall.

13. Method according to claim 7, wherein the segments and the nodes of the numerical IMD have at least one end pole, the general shape of the IMD being flattened at the level of the end pole.

14. Method according to claim 13, wherein the end pole is modelled with a first concavity during the determination of the intermediate deformation state, and is modelled with a second concavity different from the first concavity during the calculation of the mechanical equilibrium state.

15. Method according to claim 1, wherein the numerical IMD is a model of an intrasaccular cage.

16. Method according to claim 1, wherein the numerical IMD is a model of a laser-cut stent.

17. Method according to any claim 1, comprising a later step iii. of calculating a predictive local apposition of at least one part of the three-dimensional apexes of the numerical IMD on the wall model, preferably calculating a local apposition of a plurality of nodes of the numerical IMD on the wall model.

18. Method according to claim 17, wherein the numerical IMD corresponds to an IMD reference derived from a set of IMD references recorded in a database,
steps i., ii. and iii. being repeated for each reference of the set of references.

19. Computer programme product comprising code instructions for the implementation of the simulation method according to claim 1, when said code instructions are executed by a processing unit.

20. Processing unit comprising:
means for obtaining a three-dimensional wall model of a natural cavity,
means for obtaining a numerical IMD, preferably configured to generate the numerical IMD in accordance with an IMD reference derived from a database,
calculation means configured to determine an intermediate deformation state wherein the numerical IMD is deformed as a function of a shape of the wall model, while remaining included in said shape,
the calculation means being further configured to calculate a mechanical equilibrium state of the numerical IMD as a function of a mechanical behaviour of the numerical IMD and a mechanical behaviour of the wall model, the processing unit being configured to implement a simulation method according to claim 1
